(19) United States
(12) Patent Application Publication
(10) Pub. No.: US 2015/0112300 A1
(43) Pub. Date: Apr. 23, 2015

(54) METHOD FOR ENHANCED TRANS-TISSUE DELIVERY OF THERAPEUTIC SUBSTANCES

(71) Applicants: Piotr Glowacki, Umina Beach (AU); Alexander Ian McIntosh, North Turramurra (AU); Guy James Reynolds, Helensburgh (AU)

(72) Inventors: Piotr Glowacki, Umina Beach (AU); Alexander Ian McIntosh, North Turramurra (AU); Guy James Reynolds, Helensburgh (AU)

(21) Appl. No.: 14/474,060

(22) Filed: Aug. 29, 2014

(30) Foreign Application Priority Data
Oct. 9, 2013 (AU) 2013903878

(51) Int. Cl. A61K 9/00; A61K 33/16; A61N 1/44; A61K 33/42; A61N 1/05; A61K 33/00; A61K 33/06

(52) U.S. Cl. CPC A61K 9/0009; A61K 33/00; A61K 33/16; A61K 33/06; A61K 33/42; A61N 1/0568; A61N 1/44
USPC 604/501

(57) ABSTRACT

A method of enhanced trans-tissue delivery of therapeutic substances including; placing an electrode in proximity to human or animal tissue covered with material of sufficient concentration of such substances; applying a voltage signal to generate plasma, preferably Dielectric Barrier Discharge in Air, between the tissue and the electrode.

### RELATED APPLICATION

The present application claims priority from Australian Patent Application Number 2013903878, the contents of which are incorporated herein by reference.

### TECHNICAL FIELD

The present invention relates to a method of trans-tissue delivery of therapeutic substances using dielectric barrier discharge.

### BACKGROUND OF THE INVENTION

The invention takes advantage of known properties of Dielectric Barrier Discharge (DBD) and Iontophoresis.

Iontophoresis, which is also known as Electromotive Drug Administration, is a well researched technique of delivery of therapeutic substances using a small electric charge to transport the substances through a human or animal tissue.

Iontophoresis is used as a non-invasive method of delivering a medication or other bioactive agent into the body. In medicine, Iontophoresis is typically used transdermally. In dentistry, Iontophoresis can be used to facilitate transport of substances across the oral mucosa, dentin or cementum.

Iontophoresis relies on active transportation within an electric field, where electro-migration and electro-osmosis are the dominant forces in mass transport.

A significant advantage of Iontophoresis is in the transport of large, hydrophilic molecules, for example protein or peptide drugs, which are otherwise difficult to transfer through the tissue.

In conventional Iontophoresis, a power supply is used to apply a constant (DC) or alternating (AC) current, with or without a voltage bias, between the tissue and the chemical vessel, creating an ion flux.

Electro-migration of ions in the applied electric field, as well as electro-osmosis, plays a major role in transport of ions within the tissue. Electro-osmosis particularly enhances the transport of cations into the tissue due to a typical slightly alkaline pH of the skin tissue.

DBD is the electrical discharge between two electrodes, separated by an insulating dielectric barrier(s) while the electrodes are subjected to a high, alternating voltage. It has found numerous applications in science, industry, and medicine due to operating at strongly non-equilibrium conditions in various gases, including air, at atmospheric pressure, at reasonably high power levels while not necessarily using sophisticated power supplies or circuitry.

DBD plasma is typically obtained when the electrodes are separated by a gap of some millimetres and excited by alternating high voltage with frequency typically in the range 1 Hz-10 MHz. Typically in air, the DBD is formed by a large number of separate current filaments referred to as microdischarges. The microdischarges have a typical diameter in the range of 100 micrometers. These microdischarges have a complex dynamic structure and are formed by channel streamers that usually repeatedly strike at the same place as the polarity of the applied voltage changes, thus appearing as bright filaments.

In certain cases though, such as when the applied high voltage rise time is extremely short (for example dV/dt>1 kV/ns), the microfilaments may not form any stable pattern, and the discharge may appear uniform.

The microstreamers are, however, extremely short lived. The avalanche to streamer transition generally takes about 10 ns, followed by the extinction of the microstreamers. The extinction voltage of the microdischarges is not far below the voltage of their ignition. Charge accumulation on the surface of the dielectric barrier reduces the electric field at the location of a microdischarge, which results in current termination within tens of nanoseconds after breakdown. The short duration of current in the microdischarges leads to low heat dissipation and, as such, the DBD plasma remains substantially non-thermal.

As an alternative to DBD, the dielectric barrier can be replaced by a highly resistive barrier. This may be accomplished by including one or more high value resistors in the circuit, or making the electrode of a highly resistive material or a semiconductor. This mode of operation is usually called RBD (Resistive Barrier Discharge).

Importantly, the DBD is non-destructive as there is no significant rise of gas temperature and no electrical arc generation between the electrodes.

As the human or animal body is predominantly made of water, the tissue dielectric constant is sufficiently high to create DBD between the device electrode and the tissue. Most of the applied voltage appears across the DBD gap between the electrode and the tissue but the electrical current is held at safe level. Such an arrangement is usually described as Floating Electrode Dielectric Barrier Discharge (FEDBD), as a ground connection is not necessary. The low temperature, low current and non-destructive nature of the discharge renders this methodology safe for use on patients.

As a result of the phenomena described in [13], [15] and [16] above, the DBD can be safely applied to living tissues. In this case, the tissue acts as an electrode while high, alternating voltage is applied to the device electrode which is covered with dielectric. DBD is created in air between the tissue and the device electrode.

Importantly, due to the high voltage and high frequency nature of the DBD discharge, its application augments the mobility of molecules, both charged and neutral, increasing the efficiency of the delivery of the desired species through the tissue. Consequently, the method described herewith offers advantages over conventional Iontophoresis, which only facilitates the transport of ions.

Theoretical models suggest that ions move together with the surrounding water molecules when they are hydrated. The effective Stokes radius of an ion is assumed to be half of the whole size of the ion attached to the hydrating water molecule/s. When such ion vibrates due to the DBD induced alternating electric field, the interactions between the ion and water molecules are affected, resulting in a reduction in the effective Stokes radius. This leads to the increase in the diffusion efficiency.

This process affects all hydrolyzed molecules, both charged and neutral, increasing the efficiency of diffusion-based transport of the molecules.

DBD trans-tissue delivery of substances may be combined with a DC or AC based method. For example, a DC bias could be applied while the DBD is in operation to promote diffusion of specific ion species. Furthermore, as the DBD duty cycle is usually low, a DC bias could be introduced between the DBD impulses to similar effect. Moreover, the rest period between pulses may be beneficial, allowing completion of mass transport initiated by the applied voltage.

Furthermore, the waveform of the signal used to generate the DBD can be asymmetrical. For example, it could consist of very short, high-voltage pulses superimposed on direct (DC) or alternating (AC) bias voltage signal of arbitrary shape. Such superimposition may produce an electrical field of non-zero average value to promote transport of specific ion species.

Furthermore, the direct (DC) or alternating (AC) bias voltage can be applied using an auxiliary electrode, which is substantially electrically separated from the DBD electrode. Such auxiliary electrode can be touching the tissue directly, or be separated from the tissue, for example by a dielectric. The auxiliary electrode could be placed remotely from the DBD electrode, for example within the handheld part of the chassis of the device to provide electrical bias or ground connection to the patient's body, or in close proximity to the DBD electrode, for example around the DBD electrode or within the DBD electrode area of the device.

Such auxiliary electrode may be used to limit or expand the size of the treated area. The electrical field or current created by the auxiliary electrode could inhibit or promote transport of specific ions on the surface of the tissue or within the tissue.

The RBD mode can be used to apply the direct (DC) or alternating (AC) bias voltage in order to promote transport of specific ion species.

The DBD discharge will ionize the molecules of the chemicals on or adjacent to the surface of the tissue, increasing the concentration of desired species of the ions on, and near the surface of the tissue. This will create a concentration gradient of the ions, further promoting their diffusion into the tissue.

A further advantage of the DBD-enhanced process is that the alternating electric field induced by DBD discharge may increase the blood and lymphatic circulations within the tissue by dilating the relevant vessels.

A yet further advantage of combining a constant electric field with an alternating electric field such as that induced by DBD, is that it alleviates the skin irritation and burning that are known side effects of DC Iontophoresis, attributed to the accumulation of chemicals near the DC electrodes, or high levels of DC current.

### BRIEF SUMMARY OF THE INVENTION

The present invention seeks to take advantage of the properties of Dielectric Barrier Discharge to enhance the transport rate and penetration depth of therapeutic substances, both ions and electrically neutral molecules, through the skin or other relevant body interfaces such as the teeth, nails or the like, thereby making delivery of the substances through a human or animal tissue more versatile, more efficient, faster and less painful than other methods known in the art.

In accordance with the present invention, there is provided a method of trans-tissue delivery of therapeutic substances including:
a. applying the therapeutic substances to the tissue, e.g. by covering the tissue with material (liquid, solid, gas, spray, paste, foam, gel, vapor, or similar) of sufficient concentration of the therapeutic substances;
b. placing an electrode in proximity to the tissue;
c. applying a voltage signal to generate DBD between the tissue and the electrode;
d. optionally moving the electrode relative to the surface of the tissue;
e. optionally applying an auxiliary DC or AC voltage bias between the tissue and the electrode to further enhance the delivery of substances.

Preferably, the method includes applying Dielectric Barrier Discharge (DBD) between the tissue and the electrode.

Preferably, the method includes providing the electrode as a portable dielectric electrode.

DBD enhanced trans-tissue delivery of therapeutic substances has the following advantages compared with previous iontophoretic methods known in the art:
a. increased rate of transport and depth of penetration of the desired molecules, both electrically charged and neutral, into the tissue due to enhanced diffusion rate resulting from increased kinetic energy and/or the reduction in Stokes radius of hydrolyzed species;
b. increased ionization of molecules;
c. increased rate of transport due to increased blood and lymphatic circulations;
d. increased rate of transport due to dilated skin pores;
e. sterilization of the tissue;
f. possible healing enhancement for wounds, transplants, burns, scratches and similar;
g. increased blood coagulation.

### BRIEF DESCRIPTION OF THE DRAWINGS

The invention is more fully described, by way of non-limiting example only, with reference to the accompanying drawings, in which:

FIG. 1 is a substitute circuit diagram of DBD application to a tissue;

FIG. 2 is a circuit diagram for generation of DBD;

FIG. 3 is an illustration of a prototype device;

FIG. 4 is a diagrammatic representation illustrating the method of the invention;

FIG. 5 is a sketch of a concept device;

FIG. 6 illustrates a concept of DBD electrode.

### DETAILED DESCRIPTION OF THE INVENTION

FIG. 1 shows a simplified circuit diagram 1 illustrating the general principles of the invention, where: V represents a pulsed high voltage; C_{gap} is the capacitance of the gap between the electrode and the treated tissue; C_{body} represents the capacitance of the treated body; and R_{DBD} represents the resistance of DBD discharge.

As the human or animal body is predominantly made of water, the tissue dielectric constant is sufficiently high to create DBD between the device electrode and the tissue. Most of the applied voltage appears across the DBD gap between the electrode and the tissue but the electrical current is held at safe level. Such an arrangement is usually described as Floating Electrode Dielectric Barrier Discharge (FEDBD), as a ground connection is not necessary. The low temperature, low current and non-destructive nature of the discharge renders this methodology safe for use on patients.

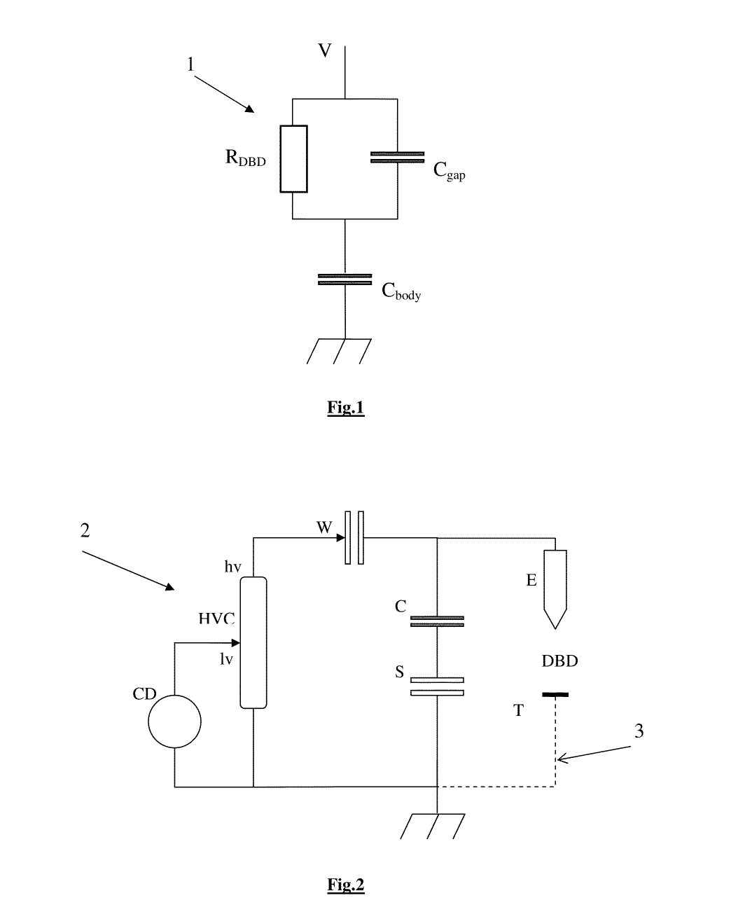

FIG. 2 illustrates an example of a circuit diagram 2 which can be used to generate and apply DBD. The circuit includes a high voltage ignition coil driver CD which controls the voltage output from a high voltage coil HVC, between a high voltage terminal hv and a low voltage terminal lv. An electrode E and capacitor C are connected in parallel to the high voltage coil though a spark gap W. A second spark gap S is provided between the capacitor and ground. T represents the treated tissue and DBD indicates the location of the dielectric barrier discharge. Grounding of tissue T is not necessary and such an optional connection 3 is shown as a dashed line.

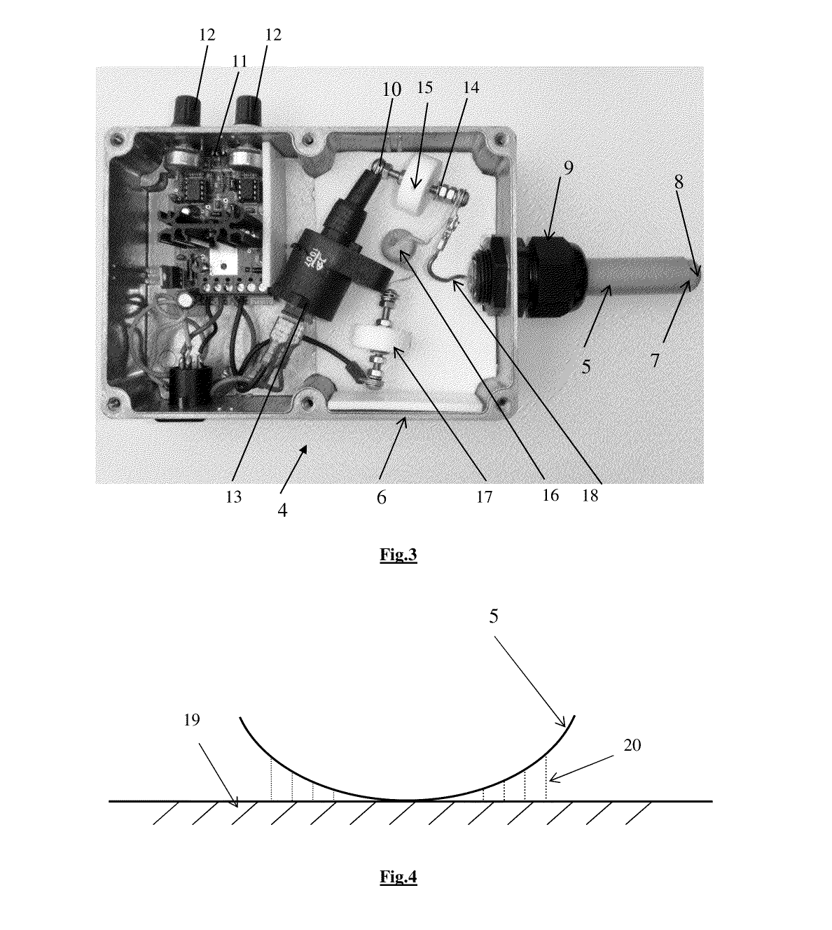

Referring now to FIG. 3, a test device 4 is shown. The device 4 includes an electrode 5 which projects externally of a housing 6. The electrode 5 is a formed of a rigid conductive member 7 covered with a dielectric covering 8. The electrode 5 can, of course, be formed as a flexible electrode.

The dielectric 8 may be formed of one or more dielectric coatings. In that regard, the dielectric may be formed of any suitable material such as ceramics, Kapton tape, quartz, glass, polypropylene or the like, with a high electric breakdown strength and low dielectric loss.

The electrode 5 is detachably mounted to the housing 6 via a screw threaded collar 9 and is electrically connected into a circuit 10, which is wired generally in accordance with FIG. 2.

More particularly, the circuit 10 is provided with a high voltage driver PCB 11, which can be adjusted or tuned via control knobs 12 to set a voltage output from high voltage coil 13. The circuit 10 has a loop 14 which includes spark gap W, as indicated by reference number 15, capacitor 16 and spark gap S, represented by reference number 17. The electrode 5 is electrically connected, via lead 18, into the circuit 10 between the spark gap 15 and the capacitor 16. The spark gaps 15, 17 are provided for the purpose of producing short energy pulses through the circuit, to the electrode 5.

The device is preferably tunable, such as by knobs 12, by adjusting the properties of the driving impulse from the high voltage coil driver, such as the amplitude, frequency, duty cycle, waveform, etc. More crudely, the spark gaps, which define the distance between the spark gap electrodes, can be adjusted.

The device 4 could also be tuned by changing the DC power supply voltage of the driver and/or coil driving voltage. Alternatively, other elements of the circuit could be varied such as the electrode, coil or capacitor, which would also affect the behavior of the device.

The device 4 has been described simply for the purpose of illustration and other circuits could instead be used, provided the circuit is capable of generating DBD when the electrode is placed in close proximity to a treated tissue.

A more advanced version of the device could contain auto-tuning hardware and/or software to optimize the discharge.

Furthermore, the direct (DC) or alternating (AC) bias voltage can be applied using an auxiliary electrode, which is, ideally, electrically separated from the DBD electrode. Such electrode can be touching the tissue directly, or be separated from the tissue, for example by a dielectric. The electrode could be placed remotely from the DBD electrode, for example within the handheld part of the chassis of the device to provide electrical bias or ground connection to the patient's body, or in close proximity to the DBD electrode, for example around the DBD electrode or within the DBD electrode area of the device.

Such auxiliary electrode may be used to limit or expand the treated area, as the electrical field or current created by the auxiliary electrode could inhibit or promote transport of specific ions on the surface of the tissue or within the tissue.

As an alternative to DBD, the dielectric barrier can be replaced by a highly resistive barrier. This may be accomplished by including one or more high value resistors in the circuit, or making the electrode of a highly resistive material or semiconductor. This mode of operation is usually called RBD (Resistive Barrier Discharge).

In particular, the RBD mode can be used to apply the direct (DC) or alternating (AC) bias voltage in order to promote or inhibit transport of specific ion species.

Importantly though, the discharge will always be a pattern of microdischarges, characteristic of DBD so that there is no spark generation which might cause discomfort or injury. As such, the invention is non-destructive and safe to use. To protect against spark generation, the electrode is coated in a dielectric material, which ensures the discharge is always DBD.

With the above in mind, the device is operated with short pulses of high voltage and long breaks in between, which means the duty cycle is low compared to a continuous sinusoidal signal. Accordingly, the power required to generate a DBD is minimal, which further renders the device safe for use on patients.

The described circuits and test device 4 have been set up to operate with filamental DBD. However, any other similar type of discharge will suffice. For example, a uniform DBD discharge or any other suitable form of atmospheric pressure glow discharge will suffice.

In either case the device 4, or at least the electrode 5, is preferably portable and hand held, allowing the treatment to be performed on any part of the body.

Turning now to FIG. 4, the methodology of the invention is explained in more detail.

As described above, DBD could be created between the device electrode and the treated tissue only when there is a gas, preferably air, present between the electrode and the tissue.

Creating such an air gap could be accomplished by keeping the distance between the electrode and the tissue sufficiently small and steady by using spacers, rollers, and the like.

Alternatively, a substantially not flat electrode can be used. FIG. 4 illustrates an example of a curved electrode 5. While electrode 5 may be directly in contact with the surface of the tissue to be treated 19 while a signal is applied from the device, DBD will be created in the air above the treated tissue, indicated by reference numeral 20, at locations adjacent to the contact area between the electrode and the tissue, where the electrode is in close proximity to the tissue.

Moreover, the electrode 5 may be substantially not flat, for example curved, dented, corrugated, pitted, perforated or similar, only locally, creating a number of areas where DBD is present.

Furthermore, the electrode 5 may consist of a number of electrodes.

To apply the DBD to a larger area, or to improve the uniformity of the treatment, a suitably sized and portable electrode 5 could be moved along, in contact or in close proximity to, the surface of the tissue 19.

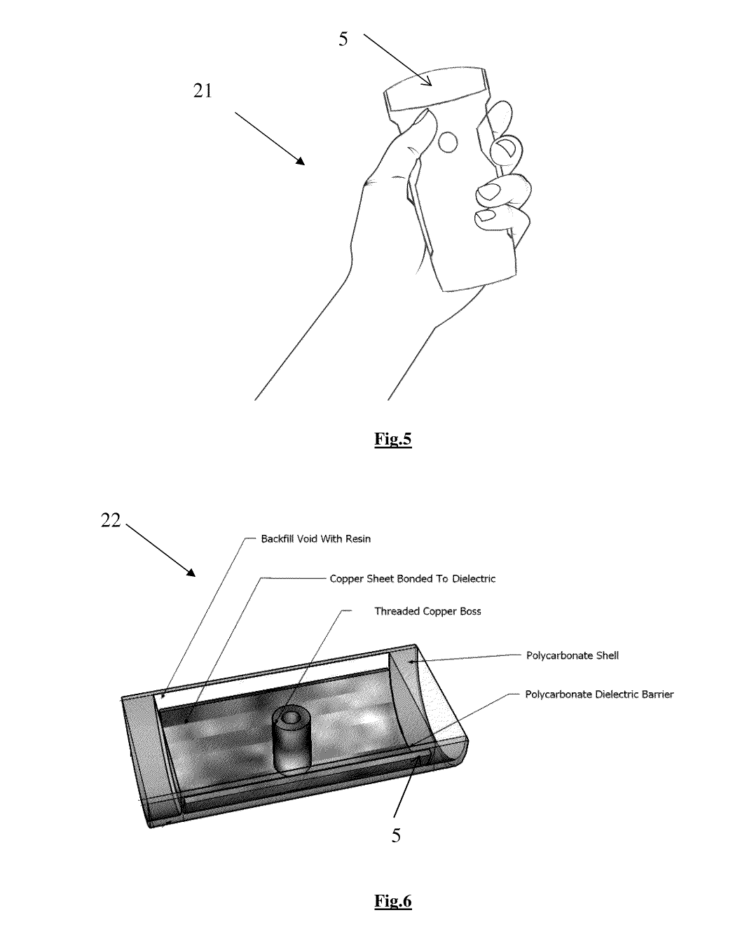

FIG. 5 illustrates a sketch of a concept, hand-held device 21 customized for trans-dermal delivery of therapeutic substances. The device electrode 5 is here shaped and sized for this purpose. The device is envisaged as fully enclosed, liquid cleanable and rechargeable in a contactless docking station. It could be operating as a stand-alone device and/or equipped with communication hardware and software for remote programming, user interface and/or data logging. It may feature disposable, single use electrodes, or electrodes suitable for sterilization by autoclave, gamma irradiation, chemical or any other method.

FIG. 6 shows a concept design 22 of the customized electrode 5 in more detail.

### EXAMPLE 1.

### DBD-Enhanced Treatment of Excessive Sweating (Hyperhidrosis)

Hyperhidrosis is a medical condition of abnormally increased sweating (perspiration), in excess of that required for regulation of body temperature. The condition can either be generalized or localized to specific parts of the body.

Hyperhidrosis is sometimes treated by Tap Water Iontophoresis (TWI). The TWI mechanism is still not completely understood. A widely accepted hypothesis is that the accumulation of hydrogen cations (H⁺) within the sweat ducts inhibits sweating. Presumably, the higher concentration of cations is the reason for the higher effectiveness of the tap water over saline solution.

The ions are driven into the skin via the pores, e.g. hair follicles or sweat gland ducts, rather than through the stratum corneum, which is the outermost layer of the epidermis. Typical sweat duct size is in 80-100 μm range for apocrine glands typically located in armpit and hand palm areas, and 30-40 μm for eccrine glands typically found elsewhere in the body.

DBD is strongly ionizing, and will increase the concentration of available hydrogen cations (H⁺) on the surface of the tissue, thus creating a concentration gradient and, subsequently, enhancing the diffusion of the cations through the tissue.

DBD may be combined with a DC bias or an AC signal to propel the hydrogen cations (H⁺) or other desired ion species into the sweat ducts.

Currently available TWI devices typically require immersing affected body parts in baths of water. The DBD enhanced method only requires application of the device to the skin, preferably wetted. If the device is fully sealed or otherwise sufficiently waterproof, the DBD could be safely and effectively applied e.g. while showering the body.

### EXAMPLE 2

### DBD-Enhanced Remineralisation of Tooth Enamel

Tooth enamel undergoes continual chemical and physical changes, which include loss of substances which are essential for maintaining the enamel integrity, e.g. Fluoride, Calcium or Phosphate. A variety of compounds are being used to help replenish these substances to the necessary levels.

If the teeth are coated with suitable mixtures or compounds containing one or more of Fluoride, Calcium and Phosphate ions, the applied DBD drives the transport of these ions into sub-surface layers of the tooth where, through the process of remineralisation, the tooth structure is repaired and strengthened, thereby helping to prevent, and potentially even reverse, the early stages of tooth decay.

### LIST OF REFERENCE SIGNS

1. Circuit;
2. Circuit;
3. Dashed line;
4. Circuit;
5. Electrode;
6. Housing;
7. Conductive member;
8. Dielectric;
9. Collar;
10. Circuit;
11. High voltage driver;
12. Knobs;
13. Coil;
14. Loop;
15. Spark gap;
16. Capacitor;
17. Spark gap;
18. Lead;
19. Tissue;
20. Microdischarge filament;
21. Sketch;
22. Design.

1. A method for enhanced trans-tissue delivery of therapeutic substances, including:
applying the substances to the tissue;
placing an electrode in proximity to the tissue;
applying a voltage signal to generate gaseous plasma between the tissue and the electrode.

2. The method of claim 1, wherein the method includes generating a dielectric barrier discharge between the tissue and the electrode.

3. The method of claim 1, wherein the method includes providing the electrode as a portable dielectric electrode.

4. The method of claim 1, wherein the method includes moving the electrode relative to the surface of the tissue.

5. A method, substantially as described with reference to the drawings and/or examples.
